(19) United States
(12) Patent Application Publication
(10) Pub. No.: US 2012/0029391 A1
(43) Pub. Date: Feb. 2, 2012

(54) BILATERAL UPPER LIMBS MOTOR RECOVERY REHABILITATION AND EVALUATION SYSTEM FOR PATIENTS WITH STROKE

(76) Inventors: Wen-Hsu Sung, Taipei City (TW); Shun-Hua Wei, Taipei City (TW); Chueh-Ho Lin, Taipei City (TW); Wen-Wei Tsai, Taipei County (TW); Yu-Da Chen, Hsinchu City (TW)

(21) Appl. No.: 12/890,295

(22) Filed: Sep. 24, 2010

(30) Foreign Application Priority Data
Jul. 30, 2010 (TW) 099125239

(51) Int. Cl. A61B 5/103; A61H 1/00

(52) U.S. Cl. 600/595; 601/33

(57) ABSTRACT

A Bilateral Upper Limbs Motor Recovery Rehabilitation and Evaluation System for Patients with Stroke which gets feedback from the feeling of body about sense of sight or auditory sense, etc to let inspector's pair of hands process forward, back, drawing and multiple actions, and let strokes can execute task training of kinematic parameter diversification about multiple strength, spped, acceleration, etc to process rehabilitation that relate to the action-status of body and estimate the status of restoration. Devices of file at least includes a pair of double-axle connecting rod structure for upper limbs, an unit for physiological signal collection, an unit for processing estimated of rehabilitation and an unit of multimedia display.

### BACKGROUND

1. Field of the Invention

The present invention relates to a bilateral upper limbs motor recovery rehabilitation and evaluation system for patients with stroke. According to the individual condition of the patient with stroke, the system provides and adjusts the functional task training mode, difficulty and duration with several kinematical parameters such as multi-angle, position, strength, speed, acceleration. Based on visual, auditory and other sensory feedback, the system provides unilateral or bilateral upper limbs joints information to assist the hemiparetic limbs of stroke patient to adjust unaffected and hemiparetic limb motor performance. The patients can also, right after the training, learn of the assessment of the upper limbs action performance by means of this bilateral or unilateral upper limbs movement training and evaluation device for patients with stroke that provides objective, quantitative and accurate result assessment.

2. Description of the Related Art

Cerebrovascular accident (CVA), commonly known as stroke, is the acute or chronic brain nerve cell necrosis caused by the ischemic or hemorrhagic infarction of the brain blood vessel which leads to the partial or total brain dysfunction, in some severe cases, even sudden death. According to the statistics, the incidence of stroke is 3 per 1,000 persions in those over 35 years of age who live in Taiwan. The cerebrovascular accident (CVA), according to the statistics in 2005 in Taiwan, has become the second major cause of death among the ten leading causes. Stroke is thus a major illness that we cannot ignore. The main effect to life function after stroke is the upper limbs motor impairment. However, study shows that only 5% of patients with stroke can gain fully functional recovery of the upper limbs while 20% of them lose completely the function.

The healthy brain cells that are not affected by the stroke can still process neural plasticity that, after enhanced training stimulus, will increase the occurrence of the cerebral cortex re-organization and facilitate the recovery of impairment. In recent years, multiple treating technologies have been developed clinically. Among them, the Compensatory Strategies, the Constraint Induce Movement Therapy (CIMT) and the Bimanual Therapy are three widely accepted modes of treatment.

The Compensatory Strategies, using unaffected compensatory performance or adjusting the surrounding equipments by unaffected extremities to increase the living independency of the patient in the early stage. However the over-reliance will deprive the hemiparetic limbs of learning from stimulus and reduce the recovery of the hemiparetic side. The Constraint Induce Movement Therapy (CIMT) has been proven to be helpful to facilitate the voluntary movement for acute stroke. However, lacking of reference of motor of the unaffected side and the training in coordination, its result of improving daily functional movements for chronic stroke patients is still challenged. The Bimanual Therapy uses daily movement of both hand movements for functional motor training and helps extending the daily functional movements. It is generally recognized as one of the most effective methods by academia and industry.

However, most of the bimanual operations focus on the overall performance movement track of the hemiparetic limb, such as the motion trajectory, the displacement of the target or the accomplishing time of a movement. Nonetheless, it is discovered in the recent studies that abnormal upper limb movements occur frequently in the stroke patients, such as unsuitable moving angle of shoulder or elbow joint.

Therefore, how to avoid unsuitable moving angle on the upper limb of stroke patients during the motor training or evaluation is one of the main problem that the academia and the industry try to solve.

### SUMMARY

Abnormal compensatory moving angle can often be observed when stroke patients practice the exercise training or during motor evaluation by the hemiparetic limbs. This invention aims at real-time analyzing the motion parameters, such as moving angle, position, strength, speed, acceleration, of the corporal joints, such as shoulder and elbow joints, of the hemipareticaffected and unaffected side of the strokes as well as the and the bilateral limbs inter-symmetry and coordination during their bilateral upper limbs motor rehabilitation training or evaluation in order to assess the motion quality of the patients.

To achieve the above objectives, the present invention provides bilateral upper limbs motor recovery rehabilitation and evaluation system for patients with stroke. The system includes at least:

A pair of double-axle connecting rod structure for upper limbs which detects the position angle of the upper limbs of the subject and converts the value detected into continuous voltage signal;

A unit for physiological signal collection which receives the voltage signal from the double-axle connecting rod structure for upper limbs, converts the voltage signal into digital signal and processes amplifying and filtering;

A unit for processing estimation of rehabilitation which receives the digital signal from the unit for physiological signal collection, analyzes and calculate the value derived from the digital signal, develops the moving position, speed, acceleration, strength and other basic kinematic parameters in order to evaluate the upper limbs recovery level of the subject and sends output of the corresponding controlling signal;

And a unit of multimedia display which receives the controlling signal of the unit for processing estimation of rehabilitation shows the upper limb position of the subject according to the instruction of the controlling signal and gives feedback of the motor information of bilateral upper limbs by the means of multimedia.

The detailed features and methods of the present invention are described thoroughly below with relevant figures.

### DETAILED DESCRIPTION

As shown in FIG. 1, FIG. 2 and FIG. 3, the Bilateral Upper Limbs Motor Recovery Rehabilitation and Evaluation includes at least:

A pair of double-axle connecting rod structure for upper limbs (1) which detects the position angle of the upper limbs as well as the value of force changing of the palm of the subject and converts the value detected into continuous voltage signal. The pair of double-axle connecting rod structure for upper limbs (1) includes at least one hemiparetic side (11) and one unaffected side (12) while the hemipareticside (11) and unaffected side (12) mechanism include at least:

An upper arm support (111) wherein the back end of the upper arm support (111) has a shoulder joint supporting frame (1111) and the front end of the upper arm support (111) has an elbow joint supporting frame (1112);

A forearm support (112) wherein the back end of the forearm support (112) couples with the elbow joint supporting frame (1112);

A hand grip (113) wherein the bottom of the hand grip (113) couples with the front end of the forearm support (112).

In addition, the hemiparetic side (11) circuit includes at least:

A first rotary potentiometer (11a), which is set at the bottom of shoulder joint supporting frame (1111) of the hemiparetic side (11) to measure the shoulder joint angle, motion time and speed of the hemiparetic side of the subject;

A second rotary potentiometer (11b), which is set at the bottom of elbow joint supporting frame (1112) of the hemiparetic side (11) to measure the elbow joint angle, motion time and speed;

A sliding potentiometer (11c), which is set at the bottom of the hand grip support (113) of the hemiparetic side (11) to allow subject to slide the whole hemiparetic side (11) to process the assigned movement and to measure the sliding angle, motion time and speed of the hemiparetic side (11);

A strain gauge (11d), which is set at the inner side of the hand grip (113) of the hemiparetic side (11) to measure the gripping force of the hemiparetic side of the subject.

Furthermore, the unaffected side (12) circuit includes at least:

A first rotary potentiometer (12a), which is set at the bottom of shoulder joint supporting frame (1111) of the unaffected side (12) to measure the shoulder joint angle, motion time and speed of the hemiparetic side of the subject;

A second rotary potentiometer (12b), which is set at the bottom of elbow joint supporting frame (1112) of the unaffected side (12) to measure the elbow joint angle, motion time and speed;

A sliding potentiometer (12c), which is set at the bottom of the hand grip support of the unaffected side (12) to allow subject to slide the whole unaffected side (12) to process the assigned movement and to measure the sliding angle, motion time and speed of the unaffected side (12);

A strain gauge (12d), which is set at the inner side of the hand grip (113) of the unaffected side (12) to measure the gripping force of the unaffected side of the subject.

Besides, the hemiparetic side (11) is shown on the left side and the unaffected side (12) on the right side in FIG. 1 and FIG. 2 of this invention only for the purpose of demonstration. It does not limit the scope of this invention only to left hemiparetic side (11) and right unaffected side (12). The left or right side can be switched according to the hemiparetic side and the unaffected side of the stroke patient.

A unit for physiological signal collection (2), which receives the voltage signal from the double-axle connecting rod structure for upper limbs (1), converts the voltage signal into digital signal and processes amplifying and filtering;

The unit for physiological signal collection (2) includes at least:

A Multi-channel analog signal collection unit (21) that converts the voltage signal received into digital signal;

A signal filter and amplifier (22) that filters and amplifies the digital signal.

A unit for processing estimation of rehabilitation (3) which receives the digital signal from the unit for physiological signal collection, analyzes and calculate the value derived from the digital signal, develops the moving position, speed, acceleration, strength and other basic kinematic parameters in order to evaluate the upper limbs recovery level of the subject and sends output of the corresponding controlling signal;

The unit for processing estimation of rehabilitation (3) includes at least:

A rehabilitation assessment software (31) that shows at least basic kinematic parameters such as moving angle, position, speed, acceleration, strength and 4 specific evaluating indexes for determining the recovery of the upper limbs of the subject. Those 4 specific evaluating indexes are:

A bilateral force symmetry value that allows identification of the force and the difference of force of the hemiparetic side and unaffected side, wherein the formulas of the bilateral force symmetry value are:

$${FSV}_{P} = \frac{{F}_{P}}{{F}_{P} + {F}_{NP}} \div BW \times 100 \% ;$$

$${FSV}_{NP} = \frac{{F}_{NP}}{{F}_{P} + {F}_{NP}} \div BW \times 100 \% ;$$

FSV_{P} is the bilateral force symmetry value of the hemiparetic side, FSV_{NP} is the bilateral force symmetry value of the unaffected side, F_{P} is the gripping force value of the hemiparetic side, F_{NP} is the gripping force value of the unaffected side, and BW is the body weight of the subject.

A bilateral force symmetry index that allows identification of the average force difference between the hemiparetic limb and unaffected limb where in the formula of the force symmetry index is:

$$FSI = {\sum }_{t = 1}^{n} \sqrt{{( \frac{{F}_{P}}{{F}_{P} + {F}_{NP}} \div BW - \frac{{F}_{P}}{{F}_{P} + {F}_{NP}} \div BW )}^{2}} ;$$

Fₚ is the gripping force of the hemiparetic limb; F_{Np} is the gripping force of the unaffected limb and BW is the body weight of the subject.

A bilateral angle symmetry value that allows identification of the change of angle of the shoulder and elbow joint on the hemiparetic side as well as the unaffected side during movement. The formula of the bilateral angle symmetry is:

$${ASV}_{S} = \frac{\sqrt{{( {A}_{PS} \div {L}_{P} \times 100 \% - {A}_{NPS} \div {L}_{NP} \times 100 \% )}^{2}}}{{A}_{PS} \div {L}_{P} \times 100 \% + {A}_{NPS} \div {L}_{NP} \times 100 \%}$$

$${ASV}_{E} = \frac{\sqrt{{( {A}_{PE} \div {L}_{P} \times 100 \% - {A}_{NPE} \div {L}_{NP} \times 100 \% )}^{2}}}{{A}_{PE} \div {L}_{P} \times 100 \% + {A}_{NPE} \div {L}_{NP} \times 100 \%}$$

L_{P} is the length of upper limb of the hemiparetic side, L_{NP} is the length of upper limb of the unaffected side, A_{PS} is the angle of shoulder joint of the hemiparetic side, A_{NPS} is the angle of shoulder joint of the unaffected side, ASV_{S} is the bilateral angle symmetry value of the shoulder joints, A_{PE} is the angle value of elbow joint of the hemiparetic side, A_{NPE} is the angle value of elbow joint of the unaffected side, ASV_{E} is the bilateral angle symmetry value of elbow joints.

A bilateral angle symmetry index that allows identification of the change and difference of the respective angles of shoulder and elbow joints of the hemiparetic and unaffected limbs. The formulas of the bilateral angle symmetry index are:

$${ASV}_{PS} = \frac{{A}_{PS}}{{A}_{PS} + {A}_{PE}} \div {L}_{P} \times 100 \% ;$$

$${ASV}_{PE} = \frac{{A}_{PE}}{{A}_{PS} + {A}_{PE}} \div {L}_{P} \times 100 \%$$

$${ASV}_{NPS} = \frac{{A}_{NPE}}{{A}_{NPS} + {A}_{NPE}} \div {L}_{NP} \times 100 \% ;$$

$${ASV}_{NPE} = \frac{{A}_{NPE}}{{A}_{NPS} + {A}_{NPE}} \div {L}_{NP} \times 100 \% ;$$

$${ASI}_{E} = \frac{\sqrt{{( {ASV}_{PE} - {ASE}_{NPE} )}^{2}}}{{ASE}_{NPS} + {ASE}_{NPE}} ;$$

$${ASI}_{S} = \frac{\sqrt{{( {ASV}_{PS} - {ASE}_{NPS} )}^{2}}}{{ASE}_{NPS} + {ASE}_{NPS}} ;$$

$$ASI = {ASI}_{E} + {ASI}_{S} ;$$

L_{P} is the length of upper limb of the hemiparetic side, L_{NP} is the length of upper limb of the unaffected side, A_{PS} is the angle of shoulder joint of the hemiparetic side, A_{NPS} is the angle of shoulder joint of the unaffected side, A_{PE} is the angle value of elbow joint of the hemiparetic side, A_{NPE} is the angle value of elbow joint of the unaffected side, ASI is the bilateral upper limbs angle symmetry index, ASI_{E} is the bilateral elbow joints angle symmetry index, ASI_{S} is the bilateral shoulder joints angle symmetry index.

Furthermore, a software (31) that provides multi-functional task training for bilateral upper limbs rehabilitation and evaluation. The task training examples, as shown in table 1, show the schematic illustrations of subject's bilateral upper limbs movement and the corresponding body movement analysis.

TABLE 1
the schematic illustrations of bilateral upper limbs rehabilitation and
evaluation movements and the corresponding body movement analysis.

| Movements | Body movement analysis |
|---|---|
| 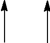 | Shoulder flexion & adduction Elbow extension |
| 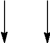 | Shoulder extension & abduction Elbow flexion |
| 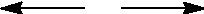 | Shoulder horizontal abduction |
| 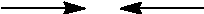 | Shoulder horizontal adduction |
| 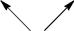 | Shoulder flexion & abduction Elbow extension |
| 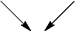 | Shoulder extension & adduction Elbow flexion |
| 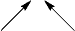 | Shoulder flexion & adduction Elbow extension |
| 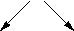 | Shoulder extension & abduction Elbow flexion |
| 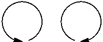 | 1. Shoulder flexion & adduction, Elbow extension 2. Shoulder flexion & abduction, Elbow extension 3. Shoulder extension & abduction, Elbow flexion 4. Shoulder extension & adduction, Elbow flexion |

A unit of multimedia display (4) that receives the controlling signal of the unit for processing estimation of rehabilitation, shows the upper limb position of the subject according to the instruction of the controlling signal, combines the kinematic signals and the instant biofeedback from the multimedia, provides information on bilateral upper limbs movement, and allows the subject to adjust the movement of hemiparetic side and unaffected side at all times. The said multimedia display unit (4) includes at least a monitor (41) and a speaker (42).

To sum up, the present invention consists of the training and evaluation of the bilateral upper limbs movements. In the training part, it includes the functional adaptation to the Rehabilitation Training Protocol and can, according to the individual situation of the stroke patient, adjust and provide corresponding training mode, difficulty and time. Also, by means of the monitor (41) and the speaker (42), it provides the stroke patient with visual and audio feedback of the bilateral upper limbs information during the rehabilitation training that allows the patient to adjust the movements on the hemiparetic and unaffected sides. In the evaluation part, the instant quantified evaluation interface allows identification of different levels of recovery of upper limb movements. Right after the training, the patient can have instant assessment of his upper limb movements with the objective and quantified evaluating results.

Although the invention has been explained in relation to its preferred embodiment, it is not used to limit the invention. It is to be understood that many other possible modifications and variations can be made by those skilled in the art without departing from the spirit and scope of the invention as hereinafter claimed.

### BRIEF DESCRIPTION OF THE DRAWINGS

FIG. 1 is the vertical view of the system of the invention.

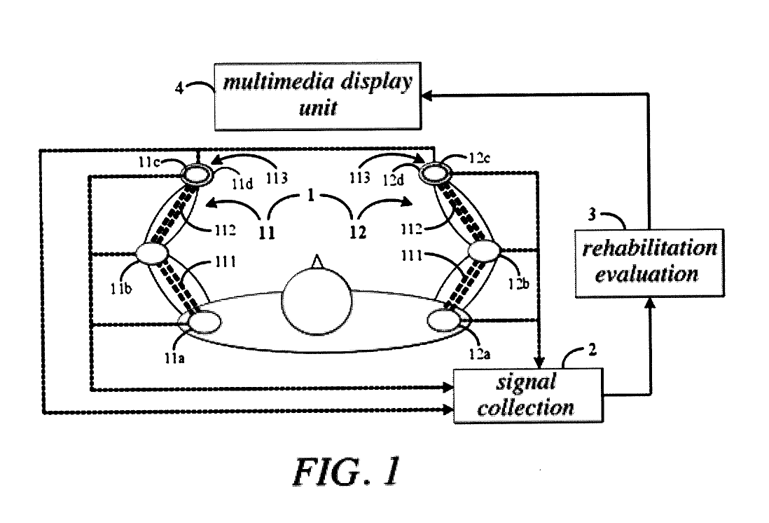

FIG. 2 is the side view of the system of the invention.

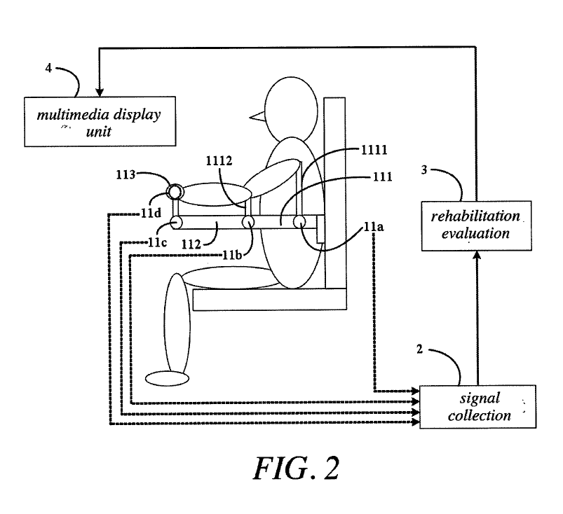

FIG. 3 is the block diagram of the circuit of the system of the invention.

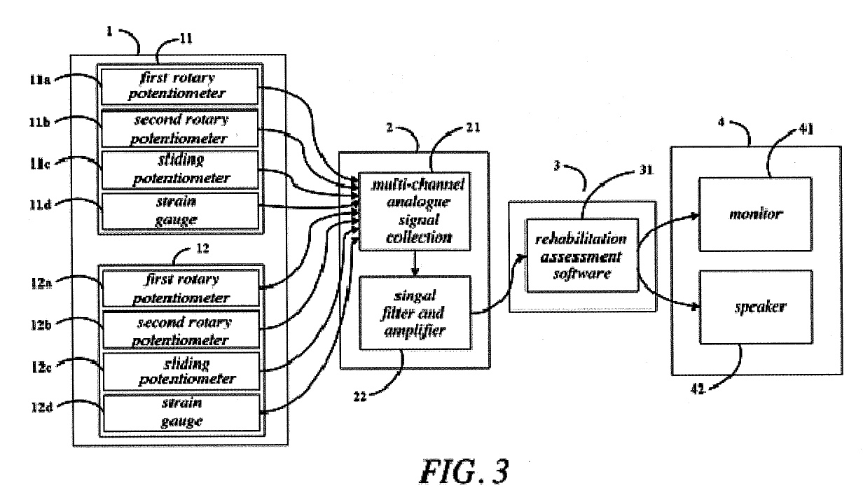

### DESCRIPTION OF MAIN COMPONENTS

| 1A | pair of double-axle connecting | | |
|---|---|---|---|
| | rod structure for | | |
| | bilateral upper limbs. | | |
| 11 | i i i | 12 | unaffected side |
| 111 | upper arm support | | |
| 1111 | shoulder joint supporting | 1112 | elbow joint supporting |
| | frame | | frame |
| 112 | forearm support | | |
| 113 | hand grip | | |
| 11a | first rotary potentiometer | 11b | second rotary potentiometer |
| 11c | sliding potentiometer | 11d | i |
| 12a | first rotary potentiometer | 12b | second rotary potentiometer |
| 12c | sliding potentiometer | 12d | strain gauge |
| 2A | unit for physiological | | |
| | signal collection | | |
| 21 | multi-channel analogue signal | | |
| | collection unit | | |
| 22 | signal filter and amplifier | | |
| 3A | unit for processing | | |
| | estimation of rehabilitation | | |
| 31 | rehabilitation assessment | | |
| | software | | |
| 4 | Multimedia display | | |
| 41 | monitor | 42 | speaker |

What is claimed is:

1. A bilateral upper limbs motor recovery rehabilitation and evaluation system for patients with stroke. The system includes at least:
A pair of double-axle connecting rod structure for upper limbs that detects the position angle of the upper limbs and the force change value of the hand grip of the subject and converts the value detected into continuous voltage signal;
A unit for physiological signal collection that receives the voltage signal from the double-axle connecting rod structure for both upper limbs, converts the voltage signal into digital signal and processes amplifying and filtering;
A unit for processing estimation of rehabilitation which receives the digital signal from the unit for physiological signal collection, analyzes and calculate the value derived from the digital signal, develops the moving position, speed, acceleration, strength and other basic kinematic parameters in order to evaluate the upper limbs recovery level of the subject and sends output of the corresponding controlling signal;
A unit of multimedia display that receives the controlling signal of the unit for processing estimation of rehabilitation, shows the upper limb position of the subject according to the instruction of the controlling signal, combines the kinematic signals and the real-timebiofeedback from the multimedia, provides information on bilateral upper limbs movement, and allows the subject to adjust the movement of hemiparetic side and unaffected side at all times.

2. The bilateral upper limbs motor recovery rehabilitation and evaluation system for patients with stroke of claim 1, wherein the pair of double-axle connecting rod structure for upper limbs includes at least one hemiparetic side and one unaffected side while the hemiparetic side and unaffected side mechanism include at least:
An upper arm support wherein the back end of the upper arm support has a shoulder joint supporting frame and the front end of the upper arm support has an elbow joint supporting frame;
A forearm support wherein the back end of the forearm support couples with the elbow joint supporting frame;
A hand grip wherein the bottom of the hand grip couples with the front end of the forearm support.

3. The bilateral upper limbs motor recovery rehabilitation and evaluation system for patients with stroke of claim 2, wherein the hemiparetic side circuit includes at least:
A first rotarypotentiometer potentiometer, which is set at the bottom of shoulder joint supporting frame of the hemiparetic side to measure the shoulder joint angle of the hemiparetic side of the subject;
A second rotary potentiometer, which is set at the bottom of elbow joint supporting frame of the hemiparetic side to measure the elbow joint angle of the hemiparetic side of subject;
A sliding potentiometer, which is set at the bottom of the hand grip support of the hemiparetic side to allow subject to slide the whole hemiparetic side to process the assigned movement and to measure the sliding angle of the hemiparetic side;
A strain gauge, which is set at the inner side of the hand grip of the hemipareticside to measure the gripping force of the hemipareticside of the subject.

4. The bilateral upper limbs motor recovery rehabilitation and evaluation system for patients with stroke of claim 2, wherein the unaffected side circuit includes at least:
A first rotary potentiometer, which is set at the bottom of shoulder joint supporting frame of the unaffected side to measure the shoulder joint angle of the unaffected side of the subject;
A second rotary potentiometer, which is set at the bottom of elbow joint supporting frame of the unaffected side to measure the elbow joint angle of the unaffected side of subject;
A sliding potentiometer, which is set at the bottom of the hand grip support of the unaffected side to allow subject to slide the whole unaffected side to process the assigned movement and to measure the sliding angle of the unaffected side;
A strain gauge, which is set at the inner side of the hand grip of the unaffected side to measure the gripping force of the unaffected side of the subject.

5. The bilateral upper limbs motor recovery rehabilitation and evaluation system for patients with stroke of claim 1, wherein the unit for physiological signal collection includes at least:
A Multi-channel analog signal collection unit that converts the voltage signal received into digital signal;
A signal filter and amplifier that filters and amplifies the digital signal.

6. The bilateral upper limbs motor recovery rehabilitation and evaluation system for patients with stroke of claim 1, wherein the rehabilitation estimation processing unit includes at least a assessment software in rehabilitation.

7. The bilateral upper limbs motor recovery rehabilitation and evaluation system for patients with stroke of claim 6, wherein the rehabilitation estimating software includes at least 4 evaluating indexes that are used to identify the recovery of the upper limbs of the subject. The 4 evaluating indexes are:
A bilateral force symmetry value that allows identification of the force and the difference of force of the hemiparetic side and unaffected side.
A bilateral force symmetry index that allows identification of the average force difference between the hemiparetic limb and unaffected limb.
A bilateral angle symmetry value that allows identification of the change of angle of the shoulder and elbow joint on the hemiparetic side as well as the unaffected side during movement.
A bilateral angle symmetry index that allows identification of the change and difference of the respective angles of shoulder and elbow joints of the hemiparetic and unaffected limbs.

8. The bilateral upper limbs motor recovery rehabilitation and evaluation system for patients with stroke of claim 7, wherein the formulas of the bilateral force symmetry value are:
$${FSV}_{P} = \frac{{F}_{P}}{{F}_{P} + {F}_{NP}} \div BW \times 100 \%$$
$${FSV}_{NP} = \frac{{F}_{NP}}{{F}_{P} + {F}_{NP}} \div BW \times 100 \%$$
FSV_{P} is the bilateral force symmetry value of the hemiparetic side, FSV_{NP} is the bilateral force symmetry value of the unaffected side, F_{P} is the gripping force value of the hemiparetic side, F_{NP} is the gripping force value of the unaffected side, and BW is the body weight of the subject.

9. The bilateral upper limbs motor recovery rehabilitation and evaluation system for patients with stroke of claim 7, wherein the formula of the force symmetry index is:
$$FSI = {\sum }_{t = 1}^{n} \sqrt{{( \frac{{F}_{P}}{{F}_{P} + {F}_{NP}} \div BW - \frac{{F}_{P}}{{F}_{P} + {F}_{NP}} \div BW )}^{2}}$$
Fₚ is the gripping force of the hemiparetic limb; F_{Np} is the gripping force of the unaffected limb and BW is the body weight of the subject.

10. The bilateral upper limbs motor recovery rehabilitation and evaluation system for patients with stroke of claim 7, wherein the formulas of the bilateral angle symmetry are:
$${ASV}_{S} = \frac{\sqrt{{( {A}_{PS} \div {L}_{P} \times 100 \% - {A}_{NPS} \div {L}_{NP} \times 100 \% )}^{2}}}{{A}_{PS} \div {L}_{P} \times 100 \% + {A}_{NPS} \div {L}_{NP} \times 100 \%}$$
$${ASV}_{E} = \frac{\sqrt{{( {A}_{PE} \div {L}_{P} \times 100 \% - {A}_{NPE} \div {L}_{NP} \times 100 \% )}^{2}}}{{A}_{PE} \div {L}_{P} \times 100 \% + {A}_{NPE} \div {L}_{NP} \times 100 \%}$$
L_{P} is the length of upper limb of the hemiparetic side, L_{NP} is the length of upper limb of the unaffected side, A_{PS} is the angle of shoulder joint of the hemiparetic side, A_{NPS} is the angle of shoulder joint of the unaffected side, ASV_{S} is the bilateral angle symmetry value of the shoulder joints, A_{PE} is the angle value of elbow joint of the hemiparetic side, A_{NPE} is the angle value of elbow joint of the unaffected side, ASV_{E} is the bilateral angle symmetry value of elbow joints.

11. The bilateral upper limbs motor recovery rehabilitation and evaluation system for patients with stroke of claim 7, wherein the formulas of the bilateral angle symmetry index are:
$${ASV}_{PS} = \frac{{A}_{PS}}{{A}_{PS} + {A}_{PE}} \div {L}_{P} \times 100 \%$$
$${ASV}_{PE} = \frac{{A}_{PE}}{{A}_{PS} + {A}_{PE}} \div {L}_{P} \times 100 \%$$
$${ASV}_{NPS} = \frac{{A}_{NPE}}{{A}_{NPS} + {A}_{NPE}} \div {L}_{NP} \times 100 \%$$
$${ASV}_{NPE} = \frac{{A}_{NPE}}{{A}_{NPS} + {A}_{NPE}} \div {L}_{NP} \times 100 \%$$
$${ASI}_{E} = \frac{\sqrt{{( {ASV}_{PE} - {ASE}_{NPE} )}^{2}}}{{ASE}_{NPS} + {ASE}_{NPE}}$$
$${ASI}_{S} = \frac{\sqrt{{( {ASV}_{PS} - {ASE}_{NPS} )}^{2}}}{{ASE}_{NPS} + {ASE}_{NPS}}$$
$$ASI = {ASI}_{E} + {ASI}_{S}$$
L_{P} is the length of upper limb of the hemiparetic side, L_{NP} is the length of upper limb of the unaffected side, A_{PS} is the angle of shoulder joint of the hemiparetic side, A_{NPS} is the angle of shoulder joint of the unaffected side, A_{PE} is the angle value of elbow joint of the hemiparetic side, A_{NPE} is the angle value of elbow joint of the unaffected side, ASI is the bilateral upper limbs angle symmetry index, ASI_{E} is the bilateral elbow joints angle symmetry index, ASI_{S} is the bilateral shoulder joints angle symmetry index.

12. The bilateral upper limbs motor recovery rehabilitation and evaluation system for patients with stroke of claim 1, wherein the unit of multimedia display includes at least a monitor and a speaker.
